(19) United States
(12) Patent Application Publication
(10) Pub. No.: US 2009/0069714 A1
(43) Pub. Date: Mar. 12, 2009

(54) System and method for measuring data for medical applications

(75) Inventors: Milton R. Eichmann, Poplar Bluff, MD (US); Gerrat Dijkman, Enschede (NL)

Correspondence Address: MCDERMOTT WILL & EMERY LLP, 600 13TH STREET, N.W., WASHINGTON, DC, 20005-3096, US

(73) Assignee: ASHLAR HOLDINGS, LLC

(21) Appl. No.: 11/898,286

(22) Filed: Sep. 11, 2007

(51) Int. Cl. A61B 5/00

(52) U.S. Cl. 600/573; 604/322

(57) ABSTRACT

The present application provides a system for measuring and storing data in an external device including a collection unit transferring fluid, a sensing unit connected to the collection unit for sensing the pressure of the fluid and converting the pressure into data, a housing assembly for housing the sensing unit, and a communication unit connected to the housing assembly receiving the data from the sensing unit and transmitting the data to the external device. The system provides a communication device for transmitting a signal to the external device having a plurality of buttons, each button corresponding to a condition, a light indicating operation of a button, and an electronic circuitry detecting the operation of the button, identifying the operation of the button as the signal, and transmitting the signal to an external device. The present application further provides a method for measuring data using the above-described system.

### FIELD OF THE INVENTION

This invention generally relates to a measuring device for medical applications. More particularly, the invention is related to a measuring device and method for measuring data of a patient and storing the data in an external device. The invention is also related to monitoring a clinical state of a patient using a communication device of the measuring system.

### BACKGROUND OF THE INVENTION

Medical devices capable of measuring various patient related data (e.g., fluid flow rates) are known in the medical field. One such device is disclosed in WO 02/07595, which provides a disposable catheter tube including at least one channel for receiving a liquid, a pressure sensor for placing in contact with the liquid, and a pressure chamber for measuring the pressure of the liquid provided with at least two ports connecting the channel and the pressure sensor. The catheter fits into a recess on a connector unit, which holds the pressure sensor enabling the catheter to contact the pressure sensor. When the catheter is used, the catheter tube is flushed and filled with water. The catheter tube is then introduced into the patient, for instance in the urinary bladder, and water is introduced into the bladder via a channel present for this purpose in the catheter tube. At this time, the pressure sensor measures the pressure of the liquid in the patient.

One advantage of such devices using a disposable catheter is increased hygiene. The disposable catheter can be supplied in a sterile state. The disposable catheters are also inexpensive. However, the conventional medical device requires a peripheral computing device, such as a computer or personal digital assistant (PDA) to transmit the measured data. Additionally, electrical wiring connects the connector unit of the conventional medical device to the peripheral equipment. Unfortunately, electrical wiring is bulky. This configuration prevents mobility of the patient. Another disadvantage of this system is that the connector unit does not have wireless capability to communicate with the doctor's computer. Thus, the patient must stay in the room near the computer.

As described in WO 04/006761, the catheter devices integrate the catheters, pressure chambers, and pressure sensors into a single cassette. FIG. 1 is a top view of a conventional measuring medical device. The measuring device 1 includes a cassette 2, which is received in a cassette holder 3. The catheters 4 transfer a liquid to pressure chambers (not shown) in the cassette 2. The measuring means of the device of FIG. 1 include three catheters 4, which are provided with three ports, i.e. a first port 5a, 5b, and 5c for connection of a catheter tube (not shown), a first venting port 7, and a second venting port 8. The catheters 4 are provided with a two-way tap 9 and a non-return valve 10 for opening and closing the catheter tube, respectively.

The catheters 4 of the conventional medical device 1 are integrated into the cassette 2. The holder 3 including the cassette 2 can be attached to the patient for ambulatory use. However, the transmitter 11 housed inside the holder 3 of the conventional medical device 1 is not part of the cassette 2. The cassette 2 is releasably connected to the holder 3 by means of a connector 13.

Recently, catheter devices have been developed to include a wireless transmitter in the connector unit. The connector unit has a plurality of recesses to receive the pressure chamber of a catheter. The absence of wires in the connector unit increases the freedom of movement of the patient to a significant degree as described in WO 04/006761. With the use of wireless communication, the patient and the peripheral equipment do not have to be located in the same room. However, the transmitter of WO 04/006761 does not provide local storage for the measured data.

The device of WO 04/006761 integrated the pressure chambers, pressure sensors, and catheters into a single cassette, which connects to the transmitter. An advantage of this system is that during ambulatory use, the measuring system is enclosed in the holder and can be worn under the patient's clothing. When the catheters are inserted inside the cassette, the cassette can be exposed to bodily fluids of the patient. A disadvantage of this system is the transmitter is housed in a separate holder, which increases the size of the measuring system.

A catheter device has been developed to include the transmitter enclosed inside the cassette during measurements. In WO 05/025415, the cassette acts as a shield for the transmitter. To ensure sterilization after the transmitter is inserted into the cassette, the cassette can be closed off to avoid contact between the bodily fluids being measured by the catheters housed in the cassette and the transmitter. The cassette may be disposable to increase sterilization.

FIG. 2 illustrates an exploded view of another conventional medical device shown and described in WO 05/025415, which includes the transmitter (not shown) enclosed inside the cassette 2 during measurements. The cassette 2 is shown in FIG. 2 having four pressure chambers 6, each of which can receive a catheter 4. Each pressure chamber 6 includes an opening into the cavity of the cassette 2, in or under which opening a pressure sensor 13 can be attached, such that the pressure sensor can measure the pressure of the bodily fluids within the pressure chamber 6. The catheters 4 and pressure sensors 13 are placed in the cassette 2 such that the cassette 2 is liquid-tight. In this case, it is possible to seal the cassette 2 off to avoid contact between the bodily fluids measured by the catheters 4 and the transmitter, connecting plate 14, and first connector 15 housed in the cassette 2. The catheters 4 are fixed to the cassette 2 and are not exchangeable.

Additionally, a chip 16 is provided inside the cassette 2 that is connected to the cassette holder 2 and to the power source to form a chip and cassette assembly. The chip 16 includes an electronic circuit with a counter or timer. The counter of the chip 16 is activated and starts timing a preset period during the operation of the medical device, for example, receiving and transmitting data obtained with the pressure sensors 13. When the pressure sensors 13 contact the pressure chamber 6, the contents of the chip 16 are read by the transmitter through the connector 15 after insertion into the cassette 2. The chip 16 performs a verification cycle to determine whether the chip and cassette assembly are compatible with transmitter. If the results of this verification cycle are compatible with the transmitter, the transmitter can read the pressure sensors 13 to obtain the desired data. After the preset period elapses, the chip 16 will stop the device and prevent reuse of the cassette 2. The chip 16 shown in FIG. 2 can also be provided with memory means in which data is stored to identify the doctor or hospital, the type of catheters connected to the cassette, and the like. This prevents unauthorized or improper use. However, the conventional medical device shown in FIG. 2 is not equipped to store the patient's data.

The cassette of WO 05/025415 is also equipped with an electronic circuit or chip to store information regarding whether the cassette has been used to measure data from the pressure sensor. The chip in the cassette is designed to allow only one use of the cassette. This prevents re-use of the cassette and possible cross-infection. However, the chip provided in WO 05/025415 does not provide local storage for the measured data.

Accordingly, there is continued interest in the development of new devices and methods for measuring medical related data of a given patient, which allows for storage of the patient's data inside the medical device. Increased wireless capability is also desired between the components of the medical device. The use of catheters having a pressure sensor housing connected to each catheter allows for easier and efficient exchange. Of particular interest would be the use of a communication device, which allows the patient to enter their clinical or urological state without using a computer or requiring assistance from medical personnel.

It is an object of the present application to solve the foregoing needs.

### SUMMARY OF INVENTION

Devices, systems, and methods are provided for measuring data of a patient using collection units, sensing units, and communication units. The collection unit or catheter transfers bodily fluids, such as blood or urine. The sensing unit or pressure sensor senses the pressure of the bodily fluids and converts this measurement into a data signal. An embodiment of the present application includes the catheter and the corresponding pressure sensor housed in its own housing. The connection between the housing and the catheters is detachable. Each housing is connected to the transmitter with a connector.

In accordance with an alternative embodiment of the present application, the transmitter can send the measurement data wirelessly to the doctor's computer or to a PDA. The computer or PDA will record, analyze, and store the measurement data. Additionally, the transmitter includes a memory card or chip inside for local storage of the measurement data. This memory card can be utilized for long term or short-term storage. For example, the memory card can be used for storage when there is no connection between the transmitter and the computer. Additionally, the memory card can also be used to input additional data into the external computer. This data can include the types of catheters used, and data specific to the patient, such as, identification, age, sex, and medical history. This feature allows the memory card to be programmed for a specific patient or a type of use. This function directs the software of the doctor's computer to perform in a custom manner depending on the specific patient or type of use stored in the programmed memory card. The memory card can also store data when the patient is ambulatory. Thus, the measurement data of the patient can be monitored during the ambulatory mode.

The system of the present application also includes a communication device that communicates with the transmitter and the computer. The communication device allows the patient to enter markers of their clinical state during their treatment. The communication device has a plurality of buttons, each button corresponding to a condition of a patient. The patient may depress a button when for example, they are feeling pain or need to urinate. Once a button is depressed, a signal is sent to the transmitter and the transmitter stores this signal locally in the memory card. The transmitter can also send the signal to the computer collecting the data. When the signal is received by the transmitter, the transmitter sends a signal back to the communication device, which displays a light to indicate that the signal has been received. The communication device can also be used to activate other devices, such as a flowmeter, for example.

Previously, the conventional device required the patient to communicate their clinical state to a medical assistant or use a PDA to enter their clinical state. The communication device of the present application allows the patient to move freely and efficiently record their clinical state. The use of the communication device also eliminates the need for a medical assistant during measurement. The communication device is a compact device, such as a remote control, unlike a PDA. Another advantage of the present application over conventional systems is that the communication device is a cheaper and simpler solution.

Another embodiment of the present application is that the wireless capability of the transmitter is extended to allow increased communication between the devices of the system. In the present application, the transmitter can talk to other wireless devices, such as the flowmeter. Therefore, the measurement data collected by the flowmeter is sent directly to the transmitter via wireless communication. After the measurement data is sent, the transmitter can store the data locally or send the data to the computing device. The patient can also press a button on the communication device to establish a connection between the transmitter and the flowmeter. This allows the patient to send measurement data at a predefined interval or when the patient experiences a change in their clinical state. The transmitter sends this data to the computer for further analysis and/or storage.

According to another embodiment of the present application, the catheter and pressure sensor assembly have wireless capability. Once the catheter and the pressure sensor assembly collect data from the patient, the measurement data can be sent to the transmitter. Because a wireless connection has been established between the catheter and pressure sensor assembly with the transmitter, the catheter and pressure sensor assembly do not need to be attached to the transmitter.

### BRIEF DESCRIPTION OF THE DRAWINGS

FIG. 1 illustrates a conventional medical device for measuring data of a patient;

FIG. 2 illustrates an exploded view of the cassette of another conventional medical device;

FIG. 3 is a view of the system for measuring data of a patient, according to an embodiment of the present application;

FIG. 4 is a view of the communication device, transmitter, and pressure sensor housings of the present application;

FIG. 5 illustrates a front view of the transmitter and pressure sensor housings, according to an embodiment of the present application;

FIG. 6 illustrates a back view of the transmitter and the pressure sensor housings, according to an embodiment of the present application;

FIG. 7 illustrates a back view of the transmitter and the connection, according to an embodiment of the present application;

FIG. 8 illustrates an enlarged view for the pressure sensor housing, according to an embodiment of the present application;

FIG. 9 illustrates an exploded view for the pressure sensor housing, according to an embodiment of the present application;

FIG. 10 illustrates the communication device in accordance with an embodiment of the present application; and

FIG. 11 illustrates a system diagram, according to an embodiment of the present application.

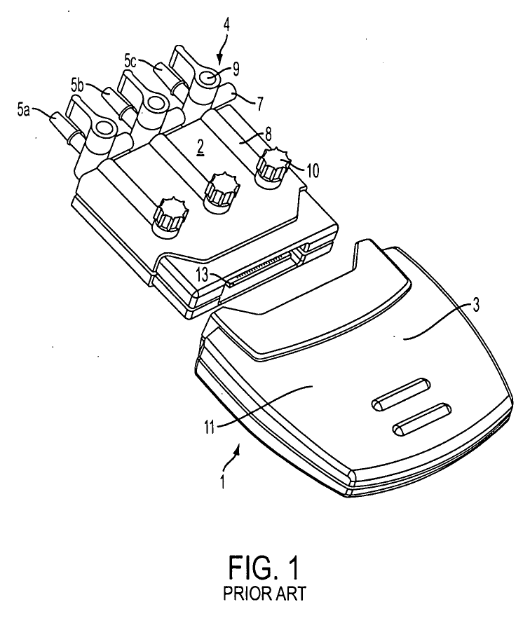

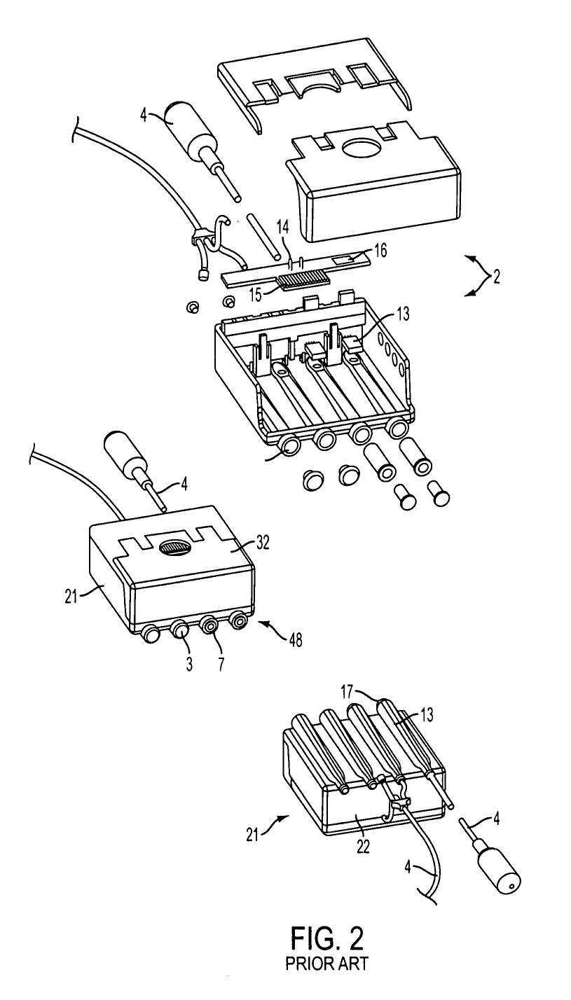

### DETAILED DESCRIPTION OF THE INVENTION

Reference will now be made in detail to the present preferred embodiments of the present application, examples of which are illustrated in the accompanying drawings, wherein like reference numerals refer to the like elements throughout. The embodiments are described below in order to explain the present invention by referring to the figures.

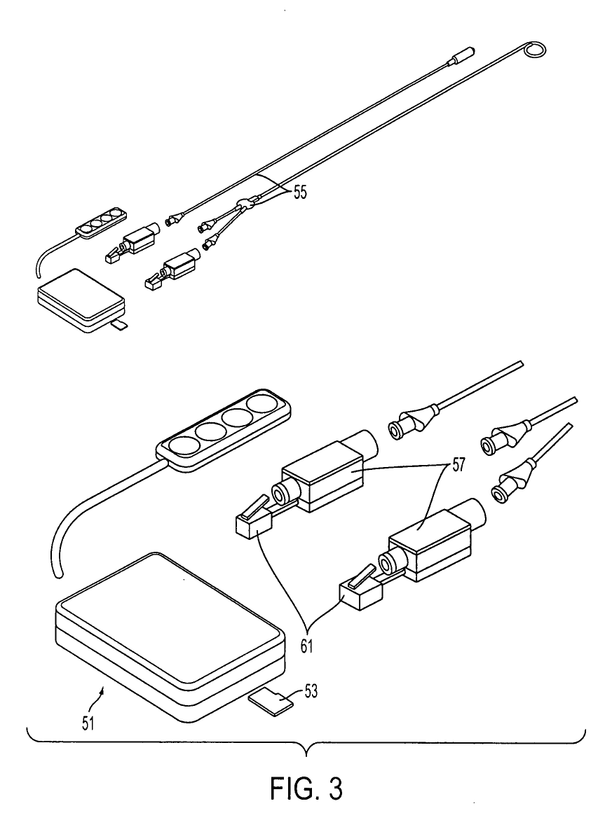

FIG. 3 is a system for measuring data of a patient, according to an exemplary embodiment of the present application. Referring to FIG. 3, in the given embodiment, the system includes at least one catheter transferring fluid of a patient, at least one pressure sensor connected to the at least one catheter for detecting pressure of the fluid and converting the pressure of the fluid into data, at least one pressure sensor housing assembly housing the at least one pressure sensor, and a transmitter releasably connected to the pressure sensor housing assembly for receiving the data from the pressure sensor and transmitting the data to an external device, such as a computer. The catheter is releasably connected to the pressure sensor housing.

Unlike conventional systems for measuring data of a patient, the transmitter 51 includes a memory card 53, which allows the transmitter 51 to compute with peripheral devices, such as the doctor's computer. The memory card 53 stores the measurement data received from the pressure sensor 59. The transmitter 51 has wireless capability to communicate with peripheral devices and transmit stored data.

Further, as shown in FIG. 3, each catheter 55 is connected to its own pressure sensor housing 57. The pressure sensor is installed inside the pressure sensor housing 57. Each pressure sensor housing 57 is connected to the transmitter 51 via a connector 61. The connectors 61 are easily attached and removed from the connections 63 found in the back of the transmitter 51 as shown in FIGS. 6 and 7. Thus, the catheters and pressure sensor housings can be replaced easily or interchanged in the present application. Another advantage of the separate pressure sensor housings is that the pressure sensor housings can be removed from the transmitter 51 after measurement. This allows the doctor or nurse to remove any pressure sensor housing individually upon completion of the measurement of the data.

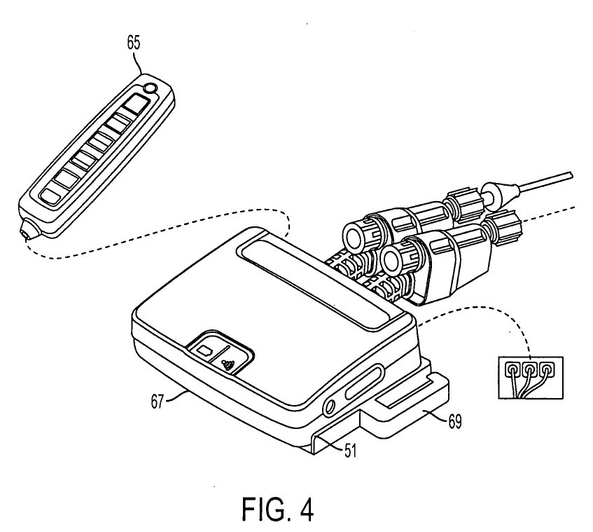

The system of the present application also includes a communication device 65 releasably connected to the transmitter 51 as shown in FIG. 4. FIGS. 4, 10, and 11 show an exemplary remote control unit as the communication device 65. However, the communication device 65 is not limited to an embodiment including a remote control. The communication device 65 is carried by the patient or inserted into their pants or jacket pocket during the ambulatory stage. The doctor or nurse can affix the transmitter 51 to the patient's underbelly using the fixation adapter 69. The fixation adapter 69 includes a self-adhesive panel on the bottom side 67 of the transmitter 51 and hooks on both sides of the transmitter 51. The fixation adapter 69 is disposable in order to maintain hygiene.

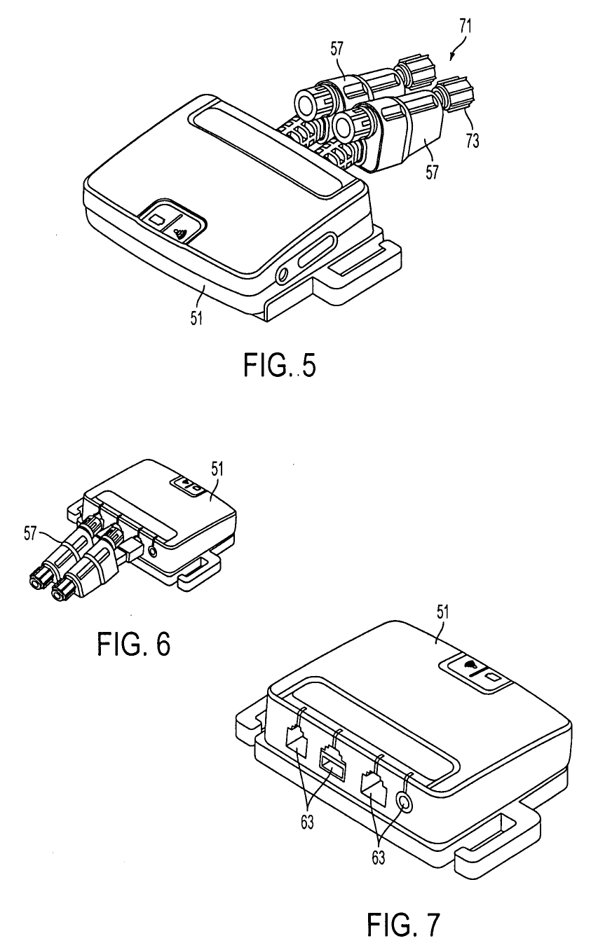

As shown in FIG. 5, in the given example, a pressure sensor housing 57 for a rectal catheter 71 and a pressure sensor housing 57 for a bladder catheter 73 are connected to the transmitter 51. The respective catheters 71 and 73 are color-coded with a blue ring or red ring, for example, to distinguish the types of catheters. Apart from the ring color, the connector for each type of housing is different. The rectal or balloon catheter connects to a housing with a 6-pole connector. The bladder catheter connects to a housing with an 8-pole connector. Both connector types are standard modular connectors, such as, for example, the ones used for telephones and computers. However, it should be understood that the catheters may be multi-lumen catheters in order to accompany both a bladder pressure part and an urethra pressure part. A pressure sensor housing for multi-lumen catheters are compatible with the connection 63 of the transmitter 51 of the present application.

FIG. 6 illustrates the back view of the transmitter 51 of the given embodiment showing two pressure sensor housings 57 connected to the transmitter 51. FIG. 7 shows the back view of the transmitter 51, which in the given embodiment includes connections for electrodes, a bladder catheter 73, a rectal catheter 71, and the communication device 65. The communication device 65 can interface with the transmitter via wireless communication technology, such as, but not limited to radio transmission signals. Importantly, the pressure sensor housing of the present application allows the doctor or nurse to detach or attach catheters individually. The transmitter and the pressure sensor housing are more compact and lighter than conventional devices. The transmitter does not contact any bodily fluid from the catheter because the pressure sensor housing transmits the measured data from the pressure sensor to the transmitter over the cable and connector assembly 77. In so doing, the cable and connector assembly increases the sterility of the system. The catheter is disposable in order to decrease the possibility of infection of the patient.

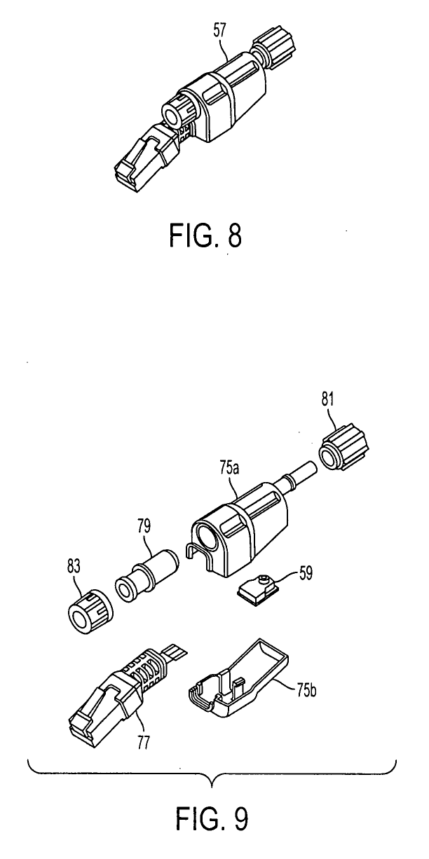

FIG. 8 is a view of an exemplary pressure sensor housing 57 of the present application after assembly. FIG. 9 is an exploded view of the pressure sensor housing 57 of FIG. 8. In each pressure sensor housing 57, there are three parts including the top cover sensor housing 75a, the bottom cover sensor housing 75b, and the cable and connector assembly 77. The wires of the cable and connector assembly 77 are connected (e.g., by soldering) to the pressure sensor 59, which allows the pressure sensor 59 to communicate with the transmitter when the cable and connector assembly 77 is inserted in a connection 63 found on the back of the transmitter. The pressure sensor 59 is then secured (e.g., glued) to an inside surface of the top cover sensor housing 75a. However, connecting of the pressure sensor housing 57 to the cable and connector assembly 77 could follow the step of the pressure sensor 59 being secured to the top cover sensor housing 75a. The checkvalve 79 is secured into the top cover sensor housing 75a. The cable and connector assembly 77 slides into the top cover sensor housing 75a. Optionally, the cable and connector assembly 77 could be fastened (e.g., glued) to the inside surface of the top cover sensor housing 75a. The bottom cover sensor housing 75b clicks onto the bottom of the top cover sensor housing 75a. This connection between the bottom cover sensor housing 75b and the top cover sensor housing 75a could be secured or made permanent with glue. The lock ring 81 has a female luer connector, which is on an end of the catheter, locking with a male luer connector, which is part of the pressure sensor housing. The lock ring 81 snaps onto the rear of the top cover sensor housing 75a and the cap 83 is screwed on the checkvalve 79. Because the catheter handling the bodily fluids is enclosed in the pressure sensor housing and the catheter does not contact the transmitter, the pressure sensor housing of the present application improves hygiene.

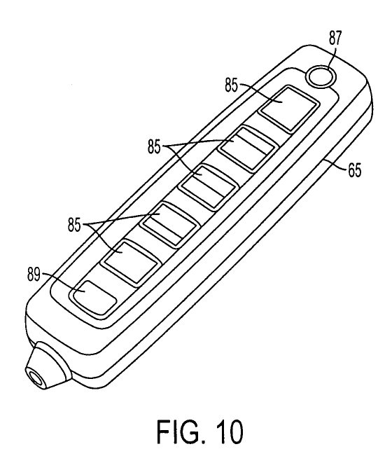

As noted above, another important aspect of the present application is the communication device 65 shown, for example, in FIG. 10. The communication device can be used when the patient is ambulatory. As shown in FIG. 10, the communication device 65 has a plurality of buttons. Each button 85 of the communication device corresponds to a predefined marker of the patient's condition, such as their clinical or urological state. For example, the buttons can be programmed to indicate that the patient is thirsty or that the patient needs to urinate. Once the patient pushes a button 85 on the communication device 65, a data signal indicative of the button pressed is sent to the transmitter 51. In the given embodiment, a light 87 on the communication device 65 is activated when the transmitter 51 receives the data signal from the communication device 65. Once the transmitter 51 receives the signal, the transmitter 51 stores the data signal in its memory chip 53 or sends the data signal to the doctor's computer. Thus, the communication device allows the patient to send predefined markers to the transmitter or computer. The communication device activates the wireless connection between the transmitter and other wireless devices, such as the flowmeter.

The communication device allows the doctor to monitor the patient and their clinical state in real-time. This feature also allows the doctor or nurse to monitor the patient in another location. The communication device of the present application also allows the patient to use the measuring device privately. The communication device decreases the need for a nurse or medical assistant to monitor the patient, which reduces cost. Another advantage of the communication device of the present application is that it allows the patient to record their current conditions during measurement when they occur.

In the given embodiment, the communication device 65 also includes a button 89 for flowmeter activation. A flowmeter (not shown) is a machine that measures urine flow collected when voiding the bladder. It is possible for the patient to turn the flowmeter on and off using this button 89 on the communication device 65. Specifically when the patient presses button 89, the connection to the flowmeter is activated. When the patient presses button 89 on the communication device 65 again, the connection to the flowmeter is terminated. Therefore, the patient can use the communication device to turn the flowmeter on and off.

If the flowmeter is equipped with a Bluetooth™ or a wireless connection, the transmitter 51 can set up a connection directly with the flowmeter. The transmitter can forward the data to the doctor's computer. The transmitter 51 can also store the data if equipped with a memory card. The communication device of the present application can also be configured to have buttons correspond to other wireless devices. By pressing a button on the communication device, the communication device can communicate with other wireless devices. This allows the patient to use the communication device to send measurement data and predefined markers to the transmitter, which has the capability to communicate wirelessly with other devices. Thus, the doctor or nurse can monitor the patient's condition without using a computer located in the patient's room. If wireless communication is used, the patient and doctor do not have to be located in the same location.

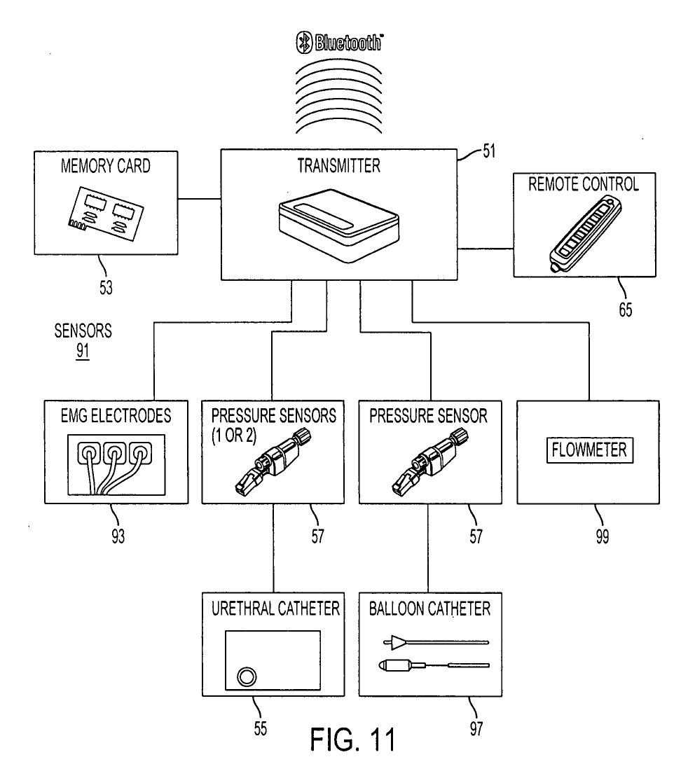

FIG. 11 illustrates an exemplary system diagram according to an embodiment of the present application. The transmitter 51 is a central device for receiving, transmitting, and storing measurement data from the sensors 91 and predefined markers for the communication device 65. The transmitter is a re-usable device, which does not contact the bodily fluids of the patient. The sensors 91, for example, include EMG electrodes 93, which are self-adhesive electrodes that register electrical signals from the muscles where they are applied to the patient. The EMG electrodes 93 transmit the electrical signals over cables to the transmitter 51. The cables of the EMG electrodes 93 are connected to the transmitter 51 in the connection 63 for electrodes shown in FIG. 7. The EMG electrodes are disposable and designed for a single use.

In an embodiment of the present application, the sensors 91 can include a pressure sensor for a single catheter or multiple catheters, such as a multi-lumen catheter. The types of catheters shown in FIG. 11 include an urethral catheter 95 and a balloon catheter 97. All of the catheters can be disposable and are intended for use in a single application. However, the catheters are not limited to the embodiment including urethral catheters and balloon catheters. The flowmeter 99 is also shown in FIG. 11. All of the sensors are connected directly to the transmitter in the present application.

The communication device 65 is connected directly to the transmitter 51. In another embodiment of the present application, the communication device communicates with the transmitter wirelessly. The memory card 53 in the transmitter 51 can store the measurement data during ambulatory use. The memory card can also be used to input additional data into the external computer. When the memory card is programmed for a specific patient or a type of use, the doctor's computer performs in a custom manner depending on the programmed memory card. The transmitter 51 collects data from the various sensors 91 and the communication device 65 and sends the data to a PDA or the doctor's computer. If the transmitter has wireless capability, the transmitter 51 transmits the measurement data wirelessly to the doctor's computer or PDA for display and analysis.

As is apparent from the above description, the present invention provides a communication device for a measuring device for medical applications. The communication device allows the patient to send indicators of their clinical state directly to the transmitter. The communication device can also activate the flowmeter connection. The communication device is less expensive than previous computing devices, which required nurses or medical assistants to manually enter the clinical data of a patient when a patient was using a catheter.

The transmitter can locally store the indicators of the patient in a memory chip or wirelessly communicate the indicators to an external computer, such as a PDA. If the catheter and the pressure sensor assembly have wireless capability, the catheter can transfer the fluid having a pressure, the pressure sensor can convert the pressure of the fluid into data, and the wireless pressure sensor assembly can send the data to the transmitter. Thus, the pressure sensor assembly does not need a connector to communicate with the transmitter.

Although a few embodiments of the present application have been shown and described, it would be appreciated by those skilled in the art that changes may be made in this embodiment without departing from the principles and spirit of the invention, the scope of which is defined in the claims and their equivalents.

1. A system for measuring and storing data in an external computer, the system comprising:
at least one catheter transferring fluid having a pressure;
at least one pressure sensor connected to the at least one catheter detecting the pressure of the fluid and converting the pressure of the fluid into data;
at least one pressure sensor housing assembly housing the at least one pressure sensor; and
a transmitter releasably connected to the at least one pressure sensor housing assembly receiving the data from the at least one pressure sensor and transmitting the data to the external computer.

2. A system as recited in claim 1, wherein the transmitter has a memory card enclosed therein storing the data.

3. A system as recited in claim 1, further comprising:
a communication device having a plurality of buttons, each button corresponding to an input signal of information, a light indicating operation of the communication device, and an electronic circuitry detecting operation of at least one button and identifying the operation of at least one button as a signal,
wherein the light is activated when at least one of the buttons are pressed, and
the electronic circuitry transmits the signal from the communication device to the transmitter.

4. A system according to claim 3, wherein the at least one catheter is releasbly connected to the at least one pressure sensor.

5. A communication device for transmitting a signal to at least one external device, the communication device comprising:
a plurality of buttons, each button corresponding to a condition;
a light indicating operation of at least one button; and
an electronic circuitry detecting the operation of the at least one button, identifying the operation of the at least one button as the signal, and transmitting the signal to the at least one external device.

6. The communication device of claim 5, wherein the plurality of buttons correspond to pain levels, urological state, and clinical state of a patient.

7. The communication device of claim 5, wherein the at least one external device is a transmitter for communicating the signal to a computer.

8. The communication device of claim 5, wherein the at least one external device is a computer for processing and storing the signal.

9. The communication device of claim 7, wherein the transmitter stores the signal in a memory card.

10. The communication device of claim 5, wherein the external device stores the signal transmitted from the communication device.

11. A system for measuring and storing data in an external device, the system comprising:
at least one collection unit collecting and transferring fluid having a pressure;
at least one sensing unit connected to the at least one collection unit detecting the pressure of the fluid and converting the pressure of the fluid into data;
at least one housing assembly housing the at least one sensing unit; and
a communication unit releasably connected to the at least one housing assembly receiving the data from the at least one sensing unit and transmitting the data to the external device.

12. A method for measuring data, the method comprising:
collecting a liquid having a pressure using at least one catheter;
detecting the pressure of the liquid using at least one pressure sensor connected to the at least one catheter;
converting the pressure of the liquid into data using the at least one pressure sensor;
transmitting the data from the at least one pressure sensor to a transmitter; and
transmitting the data from the transmitter to an external device.

13. The method according to claim 12, wherein the transmitter is releasably connected to the at least one pressure sensor.

14. The method according to claim 12, further comprising:
storing the data in a memory card enclosed in the transmitter.

15. The method according to claim 12, further comprising:
storing the data in the external device.

16. A method for transmitting a signal using a communication device having a plurality of buttons to an external device, the method comprising:
selecting at least one button, each button corresponding to an input signal of information;
detecting the selection of the at least one button;
converting the selection of the at least one button to a signal; and
transmitting the signal to the external device.

17. The method according to claim 16, further comprising:
storing the signal in a memory card enclosed in the external device.

19. A subassembly of a medical device for measuring data, the subassembly comprising:
a collection unit transferring fluid having a pressure;
a sensing unit communicating with the collection unit sensing the pressure of the fluid and converting the pressure of the fluid into data; and
a connecting unit attached to the sensing unit connecting the sensing unit to a housing having a plurality of outlets,
wherein the connecting unit is releasably connected to the plurality of outlets.

20. The subassembly of a medical device for measuring data of claim 21, wherein the collection unit and the connecting unit are affixed to an inside surface of the housing.
